(19) United States
(12) Patent Application Publication
(10) Pub. No.: US 2025/0099344 A1
(43) Pub. Date: Mar. 27, 2025

(54) SKINCARE ACTIVE INGREDIENT BOOSTERS

(71) Applicant: Mana Products, Inc., Long Island City, NY (US)

(72) Inventor: Julio Pina, Woodside, NY (US)

(21) Appl. No.: 18/893,606

(22) Filed: Sep. 23, 2024

Related U.S. Application Data
(60) Provisional application No. 63/539,789, filed on Sep. 21, 2023.

(51) Int. Cl. A61K 8/02; A61Q 19/00

(52) U.S. Cl. CPC A61K 8/022; A61Q 19/00; A61K 2800/43; A61K 2800/49; A61K 2800/591; A61K 2800/85

(57) ABSTRACT

Skincare boosters are provided. Particularly, skincare active ingredient boosters are provided. Even more particularly, the powder skincare active ingredient boosters. In some embodiments, the powder skincare active ingredient boosters may be free flowing.

### CROSS-REFERENCE TO RELATED APPLICATION

The present disclosure claims priority to Provisional Application No. 63/539,789, entitled SKINCARE ACTIVE INGREDIENT BOOSTERS, and filed 21 Sep. 2023, the content of which is hereby incorporated by reference herein in its entirety.

### FIELD OF THE INVENTION

The present disclosure relates to novel and advantageous skincare boosters. Particularly, the present disclosure relates to novel and advantageous skincare active ingredient boosters. Even more particularly, the present disclosure relates to novel and advantageous powder skincare active ingredient boosters.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Skincare products, such as gels, serums, and creams are ubiquitous. Similarly, cosmetic formulations such as primers and foundations are well-known. There are also hybrid skincare and cosmetic products, such as CC cream. However, products for converting a skincare product into a hybrid skincare and cosmetic product are not available. Similarly, products for enhancing efficacy of a skincare product are not available.

Thus, there is a need in the art for products for converting a skincare product into a hybrid skincare and cosmetic product, for converting a cosmetic formulation into a hybrid skincare and cosmetic product, or for enhancing the efficacy of a skincare product.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a skincare booster comprising a base powder and an active ingredient wherein the active ingredient is solubilized. The skincare booster may be in a loose powder formulation configured for addition to a carrier. The active ingredient may be retinal, salicylic acid, ascorbic acid, or vitamin C. In some embodiments, the skincare booster may include a second active ingredient. The base powder may comprise one or more of tapioca starch, lauroyl lysine, and miniporyl. The skincare booster may further comprise a cosmetic component that imparts a color to the booster. The skincare booster thus may have the ability to mattify, blur, and/or tint skin.

The active ingredient is solubilized in a medium. In one embodiment, the active ingredient is salicylic acid or ascorbic acid and the medium is a fermented propanediol. In another embodiment, the active ingredient is retinal and the medium is a mixture of glycerin and propanediol.

The skincare booster enables consumers to modify or enhance liquid products applied to their skin. For example, a consumer can add a skincare booster having a cosmetic effect, such as tint, to a liquid skincare item (such as serum, moisturizer, or cream) to impart cosmetic benefits to the skincare item. Alternatively, a consumer can add a skincare booster having dermatologic effect to a liquid foundation to impart skincare benefits to the foundation.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 illustrates a skincare booster delivery system, in accordance with one embodiment.

FIG. 2 illustrates a powder skincare booster, in accordance with one embodiment.

FIG. 3 is an ingredient list for a solid delivery system for a skincare active ingredient in accordance with a first embodiment.

FIG. 4 is an ingredient list for a solid delivery system for a skincare active ingredient in accordance with a second embodiment.

### DETAILED DESCRIPTION

The present disclosure relates to novel and advantageous skincare boosters. Particularly, the present disclosure relates to novel and advantageous skincare active ingredient boosters. Even more particularly, the present disclosure relates to novel and advantageous powder skincare active ingredient boosters. In some embodiments, the powder skincare active ingredient boosters are free flowing.

The skincare boosters may be used to add an active ingredient to a skincare product or cosmetic formulation. “Active ingredient” is intended to include ingredients having dermatological benefits but may also include ingredients having cosmetic benefits. Particular discussion is given herein to four embodiments of a skincare booster-one for adding retinal, one for adding salicylic acid, one for adding ascorbic acid, and one for adding vitamin C. It is to be appreciated that other active ingredients for inclusion in a skincare booster are within the scope of the present invention. Further, an active ingredient booster may include more than one active ingredient.

The skincare boosters enable consumers to modify or enhance liquid products applied to their skin. For example, a consumer can add a skincare booster having a cosmetic effect, such as tint, to a liquid skincare item (such as serum, moisturizer, or cream) to impart cosmetic benefits to the skincare item. Similarly, a consumer can add a skincare booster having a dermatologic effect to a liquid skincare item to impart other skincare benefits to the skincare item or to enhance the dermatologic effect of the skincare item. Alternatively, a consumer can add a skincare booster having dermatologic effect to a liquid foundation to impart skincare benefits to the foundation.

The skincare boosters may comprise a loose powder that can be added to a carrier. The carrier may be, for example, a serum, facial moisturizer, cream, liquid foundation, or other. In some embodiments, the combination of the active ingredient booster and carrier may produce a tinted complexion product with sheer to light coverage and having performance characteristics. The boosters have desirable characteristics of powders-such as the ability to mattify, blur, and/or tint skin-while simultaneously absorbing oil from the skin and delivering an active ingredient to the skin at an efficacious level.

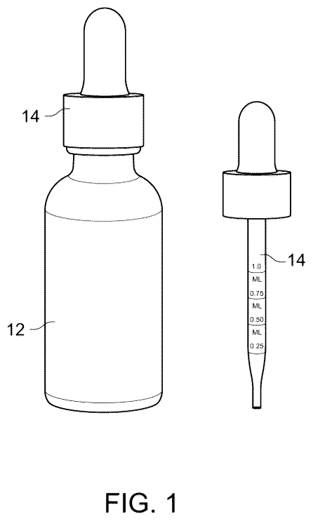

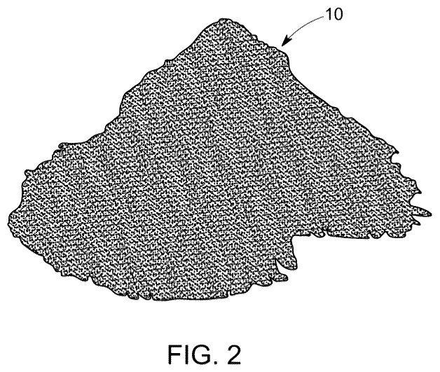

FIGS. 1 and 2 illustrates a skincare booster delivery system and skincare booster, respectively. The skincare booster may comprise a loose powder 10. The skincare booster 10 may housed in a bottle 12 having a dropper 14. A consumer may use the dropper to remove powder from the bottle 12 and put it into a carrier.

The skincare boosters comprise a base powder and a solubilized active ingredient. The base powder and solubilized active ingredient are mixed such that the base powder absorbs the liquid. In some embodiments, the skincare boosters further comprise a cosmetic component. The cosmetic component may impart a color to the skincare booster and may be provided in a plurality of colors-such as light, medium, and dark.

In one embodiment, the base powder comprises tapioca starch, lauroyl lysine, and miniporyl. Aluminum-free, non-GMO modified tapioca starch is suitable for reducing greasiness, gloss, and tack on skin. Lauroyl lysine is an amino acid powder with sweat resistant and water-resistant properties. Miniporyl is a red clover flower extract that decreases the sebum rate at the skin surface, reduces the appearance of enlarged pores, and reduces the number and size of conspicuous pores. It is to be appreciated that the base powder may comprise more or fewer ingredients than those discussed.

The active ingredient may be any suitable active ingredient. Several exemplary active ingredients are described herein. The active ingredients are solubilized in a medium or solution. The medium may be chosen based on the active ingredient. For example, the medium for the salicylic acid and ascorbic acid may be a fermented propanediol, derived from sugarcane. The medium for retinal may be a glycerin and propanediol mixture.

In one embodiment, the active ingredient is salicylic acid. Salicylic acid is effective at targeting and reducing the size and appearance of acne. The salicylic acid may be at 2%. Other percentages may alternatively be used. As is described more fully below, the salicylic acid may be solubilized in propanediol and blended into the base powder.

In yet another embodiment, the active ingredient is vitamin C. Vitamin Cis effective at brightening the skin and treating hyperpigmentation. The vitamin C may be at 1% Other percentages may alternatively be used. As is described more fully below, the vitamin C may be solubilized in propanediol and blended into the base powder.

In another embodiment, the active ingredient is retinal. The retinal may be retinaldehyde. Retinal is effective at targeting and reducing the size and appearance of acne. The retinal may be at 1%. Other percentages may alternatively be used. As is described more fully below, the retinal may be solubilized in a glycerin and propanediol mixture. In another embodiment, the active ingredient is ascorbic acid. Ascorbic acid is effective at targeting and reducing the size and appearance of acne. The ascorbic acid may be at 1%. Other percentages may alternatively be used. As is described more fully below, the ascorbic may be solubilized in propanediol and blended into the base powder.

In some embodiments, a skincare booster having a vitamin C active ingredient and a skincare booster having a salicylic acid active ingredient use the same liquid, which is primarily propanediol. In final formats, the salicylic acid may be at 2% and the vitamin C is at 1%. During production, vitamin C needs a higher ratio with propanediol; in the liquid phase, the vitamin C is at 10-12%. In one embodiment, a skincare booster having a retinal active ingredient uses a solution that has 10% glycerin.

In general, the active ingredient is solubilized in a solution to about 10-14% at the liquid phase. The solution may be, for example, a propanediol solution. Propanediol is a polyol and is effective at preventing an active ingredient from precipitating out. In some embodiments, the solution may include glycerin. By solubilizing the active ingredient in a polyol, the active ingredient is able to penetrate the skin.

The liquid phase solubilized active ingredient is blended with a powder component. The powder component may comprise blurring technology, such as mineral and synthetic microspheres, and pigments. The powder component may further comprise silica having a surface area suitable for absorbing liquid but that is not likely to dry out the skin (such as is the case with fumed silica, which additionally poses health concerns). For example, regular particle size silica may be used. The powder component absorbs the liquid while retaining the active ingredients.

The powders used in the powder component may be natural and/or synthetic with low to medium absorption values. The powders may be selected such that they are free flowing after being saturated with the liquid portion.

The resultant skincare booster, or active ingredient powder booster, may be mixed with any carrier (for example, serum, moisturizer, cream) to create a tinted skincare product. The amount of powder added to the carrier can be customized to achieve the desired amount of coverage. Tinted active ingredient powder boosters increase the efficacy of the skincare product and also add color. This converts a serum or cream to a CC serum or cream while also adding salicylic acid, retinol, vitamin C, ascorbic acid, or other active ingredient. This not only adds a cosmetic value to a skincare but also increases the efficacy of the skincare product.

In some embodiments, the active ingredient powder booster may not have a tint and may be mixed, for example, with a foundation to provide skincare properties to the foundation.

The active ingredients are solubilized in a medium. The powders utilized are natural and synthetic with low to medium absorption values such that the remain free flowing after being saturated with liquid.

The skincare boosters can be used to impart color to skincare, to add other efficacy to skincare, or to add dermatological efficacy to cosmetics.

### Method for Making Skincare Booster:

Phases A and B are utilized in making a skincare booster. Phase A comprises powders. Phase B comprises all liquids and active ingredients, such as salicylic acid, vitamin C, and retinal.

A method for making a skincare booster is outlined below. More or fewer steps may be used as would be appreciated by one skilled in the art.
Combine all powders in phase A and blend for 1 minute.
In a separate side mixing vessel, prepare phase B. The components are mixed until and homogenous.
Under propeller mixer, add Phase B to A and mix for 1 minute at high speed (200-300 rpms).
Take Phase AB and blend for 1 minute at high speed (2500-3000 rpms), stop and scrape the jar and blend another minute.

### Activated Tinted Powder Booster

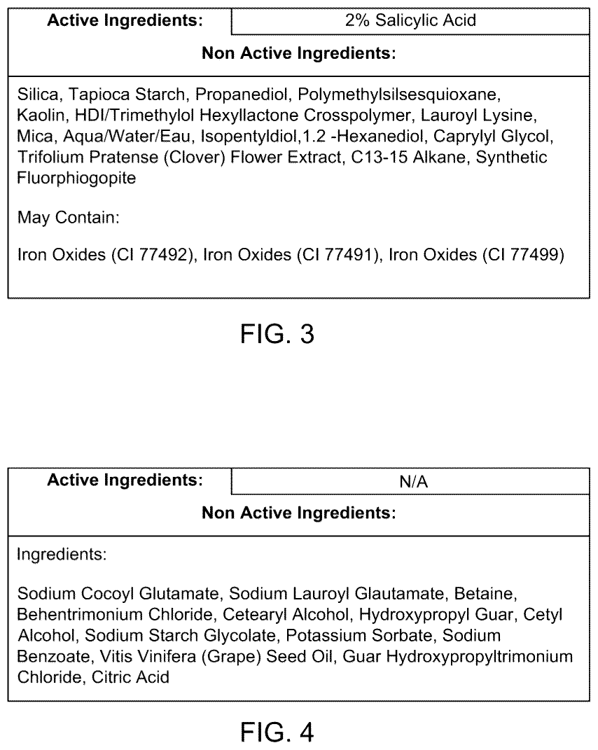

A first embodiment comprises skincare booster comprising an activated tinted booster with 2% salicylic acid. FIG. 3 illustrates an ingredient list for such booster. The tinted powder booster imparts color to a carrier, such as a skincare carrier, and enhance efficacy of the carrier. This transforms a traditional skincare product into a tinted hybrid between color cosmetics and skincare, while improving the efficacy of the skincare.

### Activated Skincare Powder Booster

FIG. 4 illustrates an ingredient list for an activated skincare powder booster for face and body.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112 (f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A skincare booster comprising:
a base powder; and
an active ingredient wherein the active ingredient is solubilized;
wherein the skincare booster is in a loose powder formulation and is configured for being added to a carrier.

2. The skincare booster of claim 1, wherein the active ingredient is retinal.

3. The skincare booster of claim 1, wherein the active ingredient is salicylic acid.

4. The skincare booster of claim 1, wherein the active ingredient is ascorbic acid.

5. The skincare booster of claim 1, wherein the active ingredient is vitamin C.

6. The skincare booster of claim 1, further comprising a second active ingredient.

7. The skincare booster of claim 1, wherein the base powder comprises one or more of tapioca starch, lauroyl lysine, and miniporyl.

8. The skincare booster of claim 1, further comprising a cosmetic component.

9. The skincare booster of claim 1, wherein the cosmetic component imparts a color to the booster.

10. The skincare booster of claim 1, wherein the skincare booster has the ability to mattify, blur, and/or tint skin.

11. The skincare booster of claim 1, wherein the active ingredient is solubilized in a medium.

12. The skincare booster of claim 11, wherein the active ingredient is salicylic acid or ascorbic acid and the medium is a fermented propanediol.

13. The skincare booster of claim 11, wherein the active ingredient is retinal and the medium is a mixture of glycerin and propanediol.
